(19) United States
(12) Patent Application Publication
(10) Pub. No.: US 2025/0242038 A1
(43) Pub. Date: Jul. 31, 2025

(54) COMPOUNDS AND METHODS FOR THE ELIMINATION OF LIVING VERTEBRATE TISSUE

(71) Applicant: Physis International LLC, Westerville, OH (US)

(72) Inventor: Frederick Oliver Cope, Farragut, TN (US)

(21) Appl. No.: 19/041,374

(22) Filed: Jan. 30, 2025

Related U.S. Application Data
(60) Provisional application No. 63/626,608, filed on Jan. 30, 2024.

(51) Int. Cl. A61K 47/61; A61K 38/17; A61K 47/54; A61K 48/00

(52) U.S. Cl. CPC A61K 47/61; A61K 38/177; A61K 47/547; A61K 47/549; A61K 48/0033

(57) ABSTRACT

These disclosures provide methods of treatment involving the elimination of tissues in vertebrate animals where such tissues may be normal or abnormal. These disclosures relate to the genetic and/or ribonucleic acid message installation of a C-type lectin receptor(s) into said tissues rendering these tissues vulnerable to cytocidal ligands. Some tissues may be preemptively obstructed by nonlethal ligands, resulting in inhibition and/or mitigation of off-target lethality by cytocidal C-type lectin receptor ligands.

### CROSS-REFERENCE TO RELATED APPLICATIONS

This application claims the benefit of U.S. Provisional Application No. 63/626,608 filed Jan. 30, 2024, the disclosures of which are hereby incorporated by reference as if fully restated herein.

### TECHNICAL FIELD

Exemplary embodiments relate to compounds and methods for the elimination, as a mode of treatment, of C-type lectin receptor-expressing cells in vertebrate animals.

### BACKGROUND AND SUMMARY OF THE INVENTION

Historically, the approach to disease therapy has been the development of pharmacological agents or other agent forms that are adapted to a specific macro- or microenvironment in which some pathological condition thrives within the body, e.g., the use of chemotherapeutic agents in treating malignant disease based on tumor tissue environments controlled by said tumor. Thus, the tactic for treatment is only within the mercy of the natural history of the pathology per se regarding the temporal and phenotypic accommodation of the treatment as may be “provided” by the disease or tissue.

Modern technologies provide the ability to specifically control macroenvironment and microenvironment elements of cells and tissues, turning the tables on localized pathologies or tissues by inducing the expression of specific macro- and micro-environmental elements, e.g., receptors, cytokines, chemokines, etc., thus rendering such pathologies vulnerable to attack with predefined pharmacological agents or other agents of choice. Such an approach provides medical practitioners the opportunity to establish a time and place of their choosing regarding therapeutic activities and eliminate pathological tissues with high specificity, providing for more reliable and durable outcomes for patients.

These disclosures relate to a compound for eliminating living vertebrate tissue and methods of treatment for a variety of diseases and disorders using the same.

One aspect of these disclosures relates to a cell-suicide inducing agent that includes Tilmanocept that has been modified though the covalent modification of its exposed carbon sites. In some embodiments, the Tilmanocept is modified through fluorination, preferably extensive fluorination. In some embodiments, the compounds include at least one bifunction linker and a C-type lectin targeting moiety. In some embodiments, the bifunctional linker is an amino-terminated sulfide linker. In some embodiments, the C-type lectin targeting moiety is mannose.

One aspect of these disclosures relates to a cell-suicide inducing agent that includes Tilmanocept that has been modified though the substitution, preferably full substitution, of DPTA with anion chelating agents. In some embodiments, the compounds include at least one bifunction linker and a C-type lectin targeting moiety. In some embodiments, the bifunctional linker is an amino-terminated sulfide linker. In some embodiments, the C-type lectin targeting moiety is mannose. In some embodiments, DPTA is substituted with an anion chelating agent, such as: macrocycles such as 1,2,3-triazole-containing calixarenes; SALTAME; and distiboranes. In some embodiments, the anion chelating agent is selected based on being able to bind monovalent anions, particularly fluorine.

One aspect of these disclosures relates to compounds that include at least one of the two cell-suicide inducing agents described above and a pharmaceutically acceptable carrier. In some embodiments, both are included. In some embodiments, at least one of the cell-suicide inducing agents further includes a stabilizing agent.

One aspect of these disclosures relates to a method of treatment using the compound described above. In some embodiments, the method includes preemptively dosing a subject (patient) with mannan, or a derivative thereof, as a blocking procedure to prevent or minimize off target localization of cell-suicide inducing agents. In some embodiments, the method includes dosing a subject with a receptor gene (carried via a viral vector, for example) and/or dosing a subject with encapsulated mRNA by a selective exposure route (e.g., inhalation, systemic injection, local/direct injection, etc.), thereby causing living vertebrate tissue (e.g., a tumor) to express C-type lectin receptor, preferably, but necessarily limited to, a functional structure of CD206. Other functional C-type lectin receptors may be chosen or combined with a functional CD206 to achieve the desired therapeutic advantage as well. In some embodiments, the method includes dosing a subject with the compound described above, which includes at least one of the two cell-suicide-inducing agents described above, thereby resulting in apoptosis of the living vertebrate tissue via fluorine toxicity.

These disclosures provide, in exemplary embodiments, without limitation, for the delivery of a select functional C-type lectin receptor of a functional gene of transcribable deoxyribonucleic acid (DNA structure) or functional messenger ribonucleic acid (mRNA) or functional short interfering nucleic acid (siRNA), any of which may be delivered per se, or as a component of an infecting agent(s) or encapsulating lipid formulation, and delivery of which may be via clinically acceptable methodology (e.g., injection, inhalation, ingestion, or transdermal (transcutaneous) or trans-tissue), and where such delivery results in the expression of said C-type lectin receptor(s) in designated target pathologies or tissue, and where the subsequent exposure of target tissues expressing a desired C-type lectin receptor are rendered vulnerable to a novel synthetic lectin receptor ligand which embodies an element that is catastrophic to the target tissue. The agent, defined as “fluoroceptin” herein, may contain an abundance of the element fluorine, known to metabolically disrupt and/or block cell energy production, resulting in cell death and elimination of the target tissue and/or disease. Pre-dosing of said cell death-inducing agent(s) provides for protective obstruction (deterring or preventing) of normal tissues expressing C-lectin type receptors from metabolic suicide induced by agents such as fluoroceptin by employing an obstructive agent such as mannan, a mannan derivative, a mannan analog(s) or other C-type lectin receptor-recognized carbohydrate polymer(s) acting as a pseudo-ligand(s) competitive or noncompetitive inhibitor(s) of suicide-inducing ligand(s), resulting in the preservation of normal functioning, nontarget tissue.

These disclosures provides for the installation, intracellular expression, and the superficial display of selected functional C-type lectin transmembrane receptors in tissues of vertebrate animals via selected receptor gene transfer and/or messenger ribonucleic acid installation, wherein such transfer and C-type lectin receptor display renders said target tissues vulnerable to confrontation by synthetic C-lectin ligands which embody a cytologically lethal element(s), and where such ligands are internalized into the cell by the C-lectin transmembrane receptor(s) resulting in exposure of the intracellular environment to said ligands, inflicting intracellular disruption and the induction of cell death by metabolic catastrophe leading to apoptosis and/or alternative non-DNA fragmenting cell death process and/or frank lysis. These disclosures, as a component of the complete treatment method, embodies a preemptive protective obstruction of lethal ligand(s) in normal tissues or off-target tissues by employing a non-lethal competitive ligand that mitigates or eliminates off target effects of the lethal ligands, thus improving the therapeutic index of said lethal C-type lectin receptor ligands.

### BRIEF DESCRIPTION OF THE DRAWINGS

FIG. 1 is an illustration showing the general structure of Tilmanocept per se;

FIGS. 2A-2C are illustrations of linkers attached to the core glucose elements of Tilmanocept with vulnerable fluorination sites;

FIG. 3 is an illustration of linkers attached to core glucose elements;

FIG. 4 is an illustration of linkers attached to the core glucose elements with fluorinated linker sites;

FIG. 5 is an illustration of a 1,2,3-triazole-containing calixarenes;

FIG. 6 is an illustration of various lectin receptors;

FIG. 7 is a visualization of the CD206 gene;

FIG. 8 is a visualization of the CD208 gene obtained from the NCBI (National Center for Biotechnology) Gene database;

FIG. 9 is a visualization of the CD209 gene obtained from the NCBI Gene database;

FIG. 10 is a visualization of a CD280 gene obtained from the NCBI Gene database;

FIG. 11 is an illustration of modifying compounds;

FIG. 12 is an illustration of standard and modified mRNA-modifying nucleotides;

FIG. 13 is an illustration showing transfection, transportation, translocation and expression of a gene (DNA) or a message (mRNA) leading to protein (selected C-type lectin receptor) production or siRNA leading to protein (C-type lectin receptor) production inhibition;

FIG. 14 is an illustration of mannan and various derivatives thereof; and

FIG. 15 is an illustration showing the overall schema of an embodiment of the clinical operation of the present invention.

### DETAILED DESCRIPTION OF EXEMPLARY EMBODIMENT(S)

The following detailed description refers to the accompanying drawings, which illustrate specific examples described by the disclosure. Other examples having different structures and operations do not depart from the scope of the present disclosure. Like reference numerals may refer to the same feature, element, or component in the different drawings.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Tilmanocept is a mannosylaminodextran chemically defined as:
dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes.

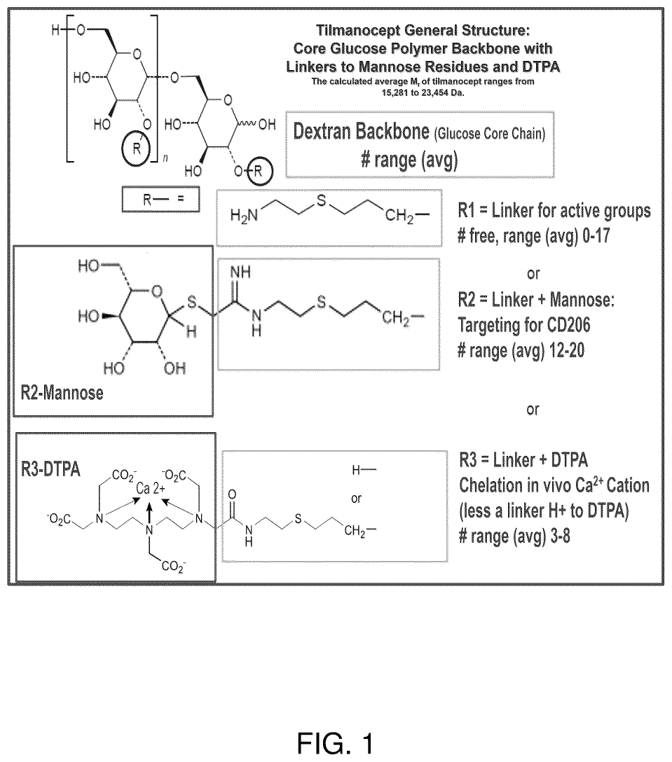

FIG. 1 depicts the general structure of Tilmanocept as it is currently known, which for the purposes of the present disclosure may be referred to as “Tilmanocept per se”. As shown, Tilmanocept per se includes a dextran backbone that comprises a plurality of core glucose elements. Linkers, such as the amino-terminated sulfide linker O(CH₂)₃S(CH₂)₂NH₂), may be attached to the core glucose elements to provide binding sites for various active groups (R1) and/or to enable the conjugation of various moieties (R2, R3).

Skilled artisans will appreciate that linkers, including generally linear bifunctional linkers such as O(CH₂)₃S(CH₂)₂NH₂), have one or more distal ends (distal relative to the backbone molecule) where a target molecule may be conjugated or otherwise attached to the linker. The distal ends are sometimes referred to as the “terminus” or “end group” or “reactive site”.

An important type of moiety which can be conjugated includes C-type lectin targeting moieties which facilitate the delivery of Tilmanocept (in some form) to C-type lectin receptors in a subject. This is represented in FIG. 1 as mannose (R2). The primary target for mannose is the C-type lectin receptor CD206.

As indicated in FIG. 1, another type of moiety which may be conjugated is an ion chelator. This is represented in FIG. 1 as diethylenetriamine penta-acetic acid (DTPA) (R3).

It is noted that the linkers with conjugated mannose and the linkers with conjugated DTPA may be attached to different glucose elements of the Tilmanocept dextran backbone.

One aspect of the present invention relates to methods of modifying Tilmanocept per se to produce cell-suicide inducing agents. Broadly, these methods involve adding fluorine at various locations to produce a receptor targeted ligand based on Tilmanocept that is capable of traversing the cellular membrane of a C-type lectin expressing cell via a C-type lectin receptor in order to deliver a sufficient amount of fluorine to induce cellular suicide by fluorine toxicity.

Fluorosis can result in mitochondrial disfunction, which can negatively impact organs and tissues in a subject. When fluoride invades into cells and damages mitochondria, that can cause decreased activity of mitochondrial related enzymes, a weakening of protein expression, damage to the respiratory chain, excessive fission, disturbance of fusion, disorder of calcium regulation, a decrease of intracellular ATP, and the accumulation of reactive oxygen species. Furthermore, the decrease of mitochondrial membrane potential can at the same time lead to the release of cytochrome c, causing a series of caspase cascade reactions and resulting in apoptosis.

The present invention provides at least two methods of modifying Tilmanocept per se—a first method that generally involves the covalent modification of exposed carbon sites on the Tilmanocept per se molecule; and a second method that generally involves substituting DPTA with anion chelating agents. The product of the first method may be referred to as “Tilmanocept-F” (TIL-F) herein; the product of the second method may be referred to as “Fluoroceptin” (FCN) herein. These methods, separately or together, are aspects of the present invention. These cell-suicide inducing agents and the compounds that incorporate them are considered other aspects of the present invention. It is noted that compounds according to the present invention can include one or both of these cell-suicide inducing agents, as well as various additives (e.g., stabilizing agent).

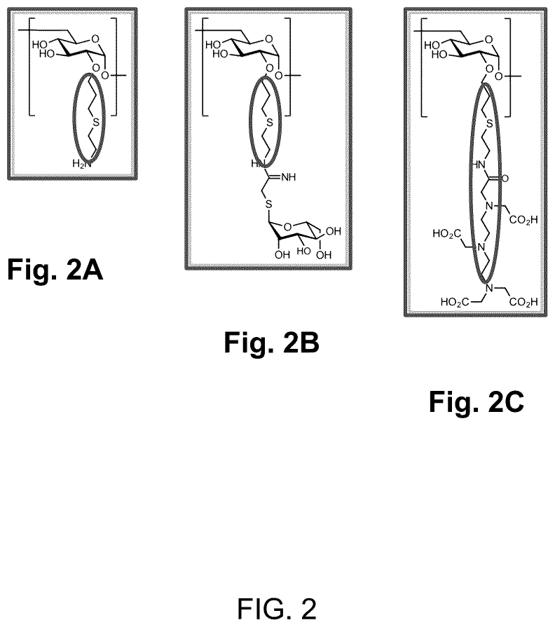

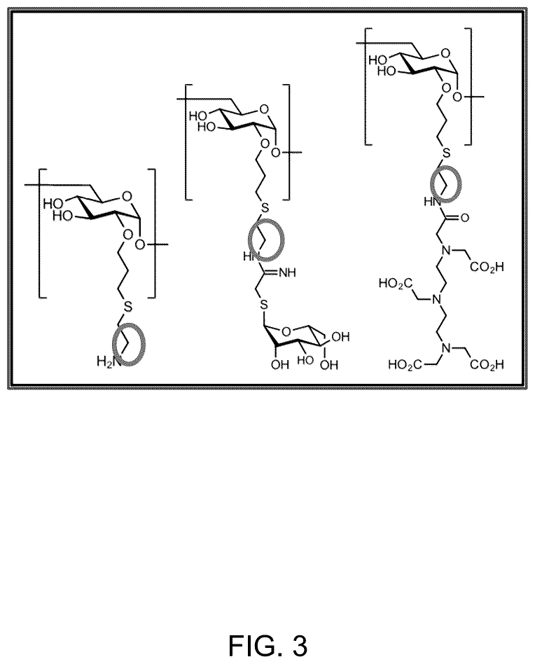

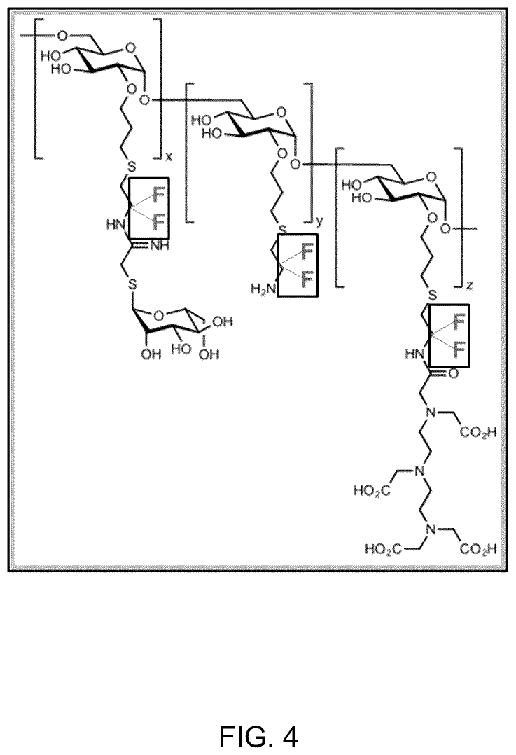

The first method entails fluorinating a plurality of exposed carbon sites on the Tilmanocept per se molecule. FIGS. 2A-2C and FIG. 3 identify some carbon sites which may be modified as described (circled in green and blue, respectively). It should be understood that these carbon sites are not meant to be limiting, as there may be other carbon sites that are fluorinated as well. FIG. 4 shows an example of the resultant TIL-F. It is noted that some carbon sites may be polyfluorinated though this process.

The primary carbons (which may be primary carbocations) of the linker molecules (circled in FIGS. 2A-2C and FIG. 3), which are disposed near the distal ends of the linker molecules, can be fluorinated up to the maximum extent possible (either with one or two fluoride atoms each). In exemplary embodiments, the compound may include TIL-F having a total number of attached fluoride atoms ranging from 1-20, or more preferably from 5 to 50, or even more preferably from 25 to 250.

In embodiments where the compound includes TIL-F having both fluoride atoms and chelated cations attached (e.g., via DTPA), the total number of fluoride atoms may range from 1 to 20 and the total number of chelated cations may range from between 5 to 50; or more preferably, the total number of fluoride atoms may range from 25 to 250 and the total number of chelated cations may range from between 1 to 20; or even more preferably, the total number of fluoride atoms may range from 5 to 50 and the total number of chelated cations may range from between 25 to 250. Other ranges may be utilized.

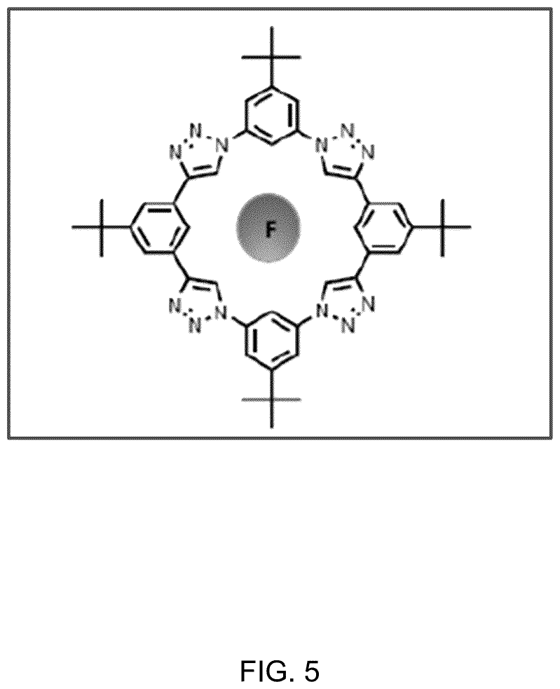

The second method entails the full substitution of DTPA with an anion chelating agent. The anion chelating agent may be used to bind certain anions, particularly, but not limited to, monovalent anions such as fluorine. Preferred anion chelating agents for this role include macrocycles such as 1,2,3-triazole-containing calixarenes (FIG. 5). These molecules define a center hydrophobic cavity that can hold smaller ions like fluorine. Other anion chelating agents which may be suitable include, for example without limitation, salicylidene-tris(aminomethyl)ethane (SALTAME) and distiboranes. Distiboranes can be accessed by the reaction of the o-C₆F₄(SbPh₂)₂ with o-chloranil or octafluorophenanthra-9,10-quinone. This results in distiboranes with the general formula o-C₆F₄(SbPh₂(diolate))₂, with the diolate being tetrachlorocatecholate for octafluorophenanthrene-9,10-diolate. These chelators may be linked through a variety of organic chains, such as any of the bifunctional linkers previously described in the present disclosure.

In exemplary embodiments, the linkers described above may further support the attachment of stabilizing groups. As used herein, the term “stabilizing group” may be used to refer to any moiety capable of preventing or at least substantially inhibiting chemical degradation. This may include, but is not limited to, methyl acrylate or N-ethylmaleimide.

The cell-suicide inducing agents described above—TIL-F and FCN—exemplify a class of C-type ligands which may be received by a C-type lectin receptor. These C-type ligands are either constructs or include construct elements adapted from a multitude of ligands to provide the necessary affinities for a chosen C-type ligand receptor. Table 1, below, provides a list of specific carbohydrate constructs which define specificity and affinity for certain C-type ligand receptors.

TABLE 1
C-Type Lectin Receptors
C-type lectin-like receptors [symbol] DAMPs.

| | Gene | | DAMP | Functional | Associated |
|---|---|---|---|---|---|
| | name | Expression | ligand/s | effects | diseases |
| [symbol] | [symbol] | [symbol] | [symbol] | [symbol] | [symbol] |
| LGX-1 | [symbol] | [symbol] | [symbol] | [symbol] | hypertension [symbol] |
| | | | [symbol] | | metabolic symptoms, coronary |
| | | | | | artery disease and cancers |
| [symbol] | [symbol] | [symbol] | [symbol] | [symbol] | Promotes obesoty, rheutatoid |
| [symbol] | [symbol] | | [symbol] | [symbol] | arthritis, allergic contact |
| | | | [symbol] | [symbol] | dermatitis, ischemic stroke, |
| | | | | | traumatic brain injury, hepatitis, |
| | | | | | [symbol] |
| [symbol] | [symbol] | [symbol] | Mono-sodium | [symbol] | [symbol] |
| [symbol] | [symbol] | [symbol] | urate crystals | [symbol] | |
| [symbol] | | | | [symbol] | |
| | | | | [symbol] | |
| | | | | [symbol] | |
| | | | | ↓FC6, IL-8 | |
| | | | | [symbol] | |
| [symbol] | [symbol] | [symbol] | F-actis | [symbol] | Promotes atherosclerosis and |
| [symbol] | [symbol] | [symbol] | | [symbol] | pancreatitis |
| | | [symbol] | | ↓ MIP-2 and excessive | |
| | | | | neurophil infiltration | |

[symbol]

[symbol]

indicates data missing or illegible when filed

C-type lectin-like receptors are typically type II transmembrane glycoproteins with an N-terminal cytoplasmic domain, a transmembrane domain followed by a stalk domain, and an extracellular C-type lectin-like domain at the C-terminus. The CTLD comprises two α-helices and two antiparallel B-sheets stabilized by two or three conserved intramolecular disulfide bonds and several connecting loops involved in ligand recognition. In addition, CTLRs usually dimerize in a homo- or hetero-manner via the cysteine residue present in their stalk region.

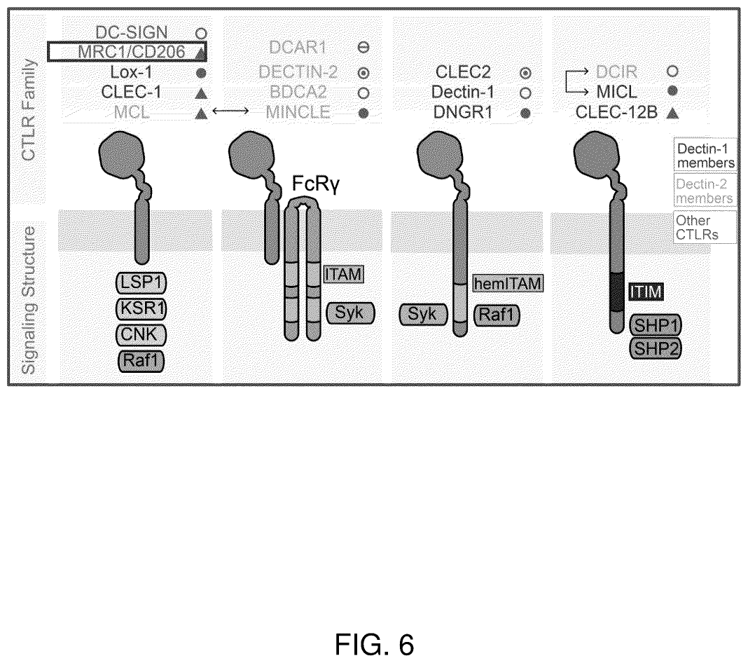

Members of the C-type lectin-like family include the NK cell receptors from the CLEC2 family (i.e., human CD69, KACL, AICL, and LLT1, or murine Clr ligands), the KLR family (i.e., human NKR-P1 or murine Ly49), and the standalone activating NKG2D receptor and various CD94/NKG2x heterodimeric receptors. Provided in FIG. 6 is an exemplary list of these C-type lectin receptors.

CD206 (identified by the red box in FIG. 6) is the primary receptor target for TIL-F and FCN. The structures of TIL-F and FCN are particularly well suited to accommodate this receptor due to embodying high mannose residence. Installation of CD206 may render the tissue or cells vulnerable to the induction of cell suicide by fluorine-carrying C-type lectin ligands via mitochondrial disruption due to fluorine toxicity.

In exemplary embodiments, transfection capabilities provide C-lectin receptor expression of a selected receptor in the target tissue (namely, but not necessarily limited to, CD206). Expression of a selected receptor may be accomplished in at least two ways:
1. via the insertion of the receptor gene, its induction, transcription (mRNA production), mRNA translation, protein expression, and cell membrane posting (superficial display); or
2. via the delivery and uptake of the target tissue of the specific message (mRNA) for the designated receptor, and mRNA translation, protein expression and cell membrane posting (superficial display) of that receptor in the target tissue.

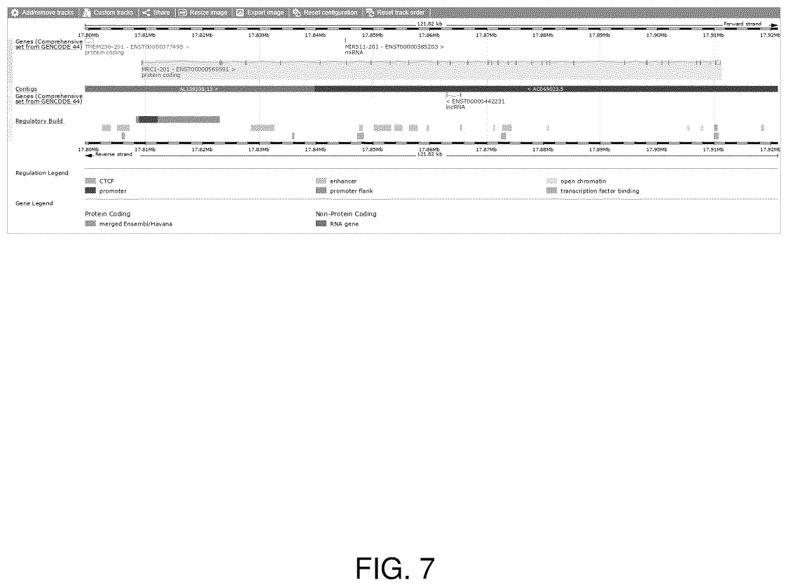

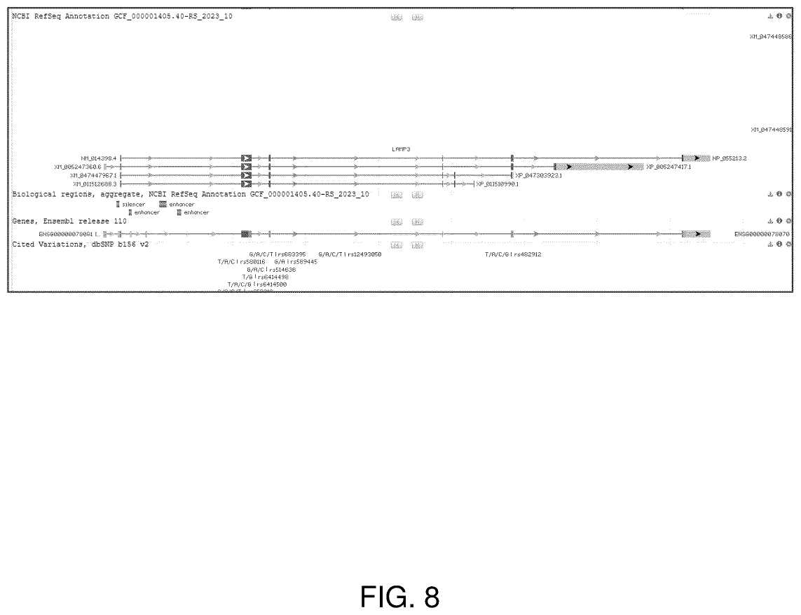

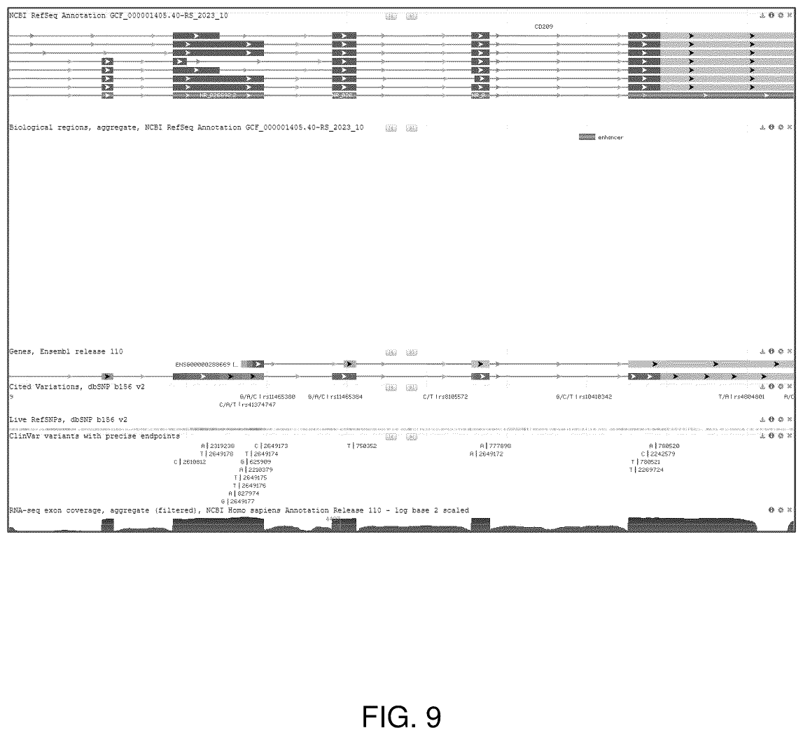

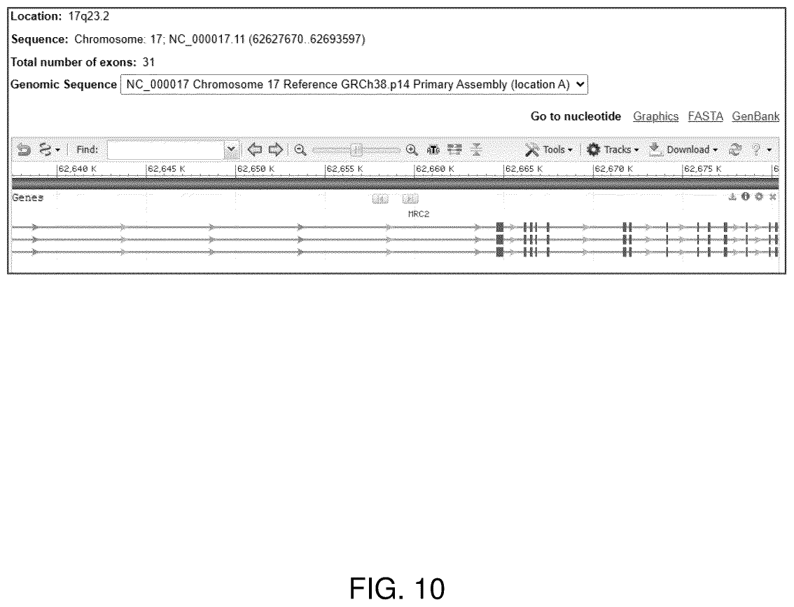

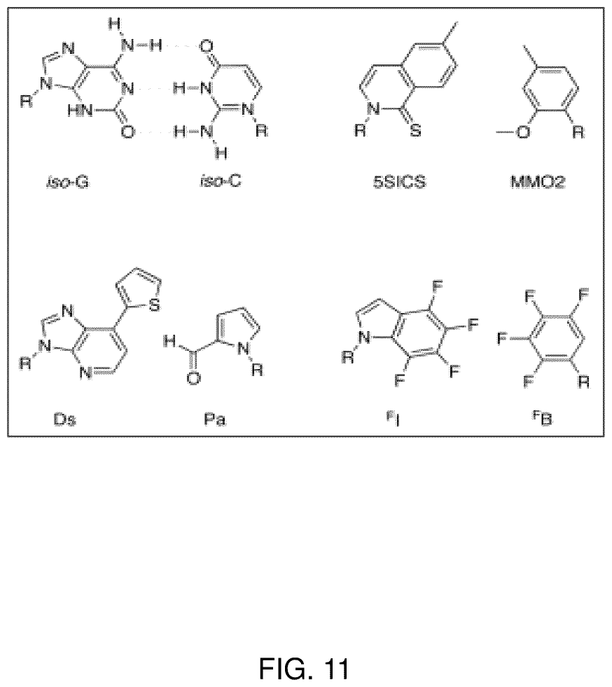

Regarding the first way of expressing a cell (i.e., insertion of the receptor gene), FIGS. 7-10 show several exemplary receptor genes which may be suitable for this role. It is noted that the gene that is inserted may be modified with one or more of the modifying compounds shown in FIG. 11 and/or other nucleoside analogs, nucleoside derivatives (including nucleotides), modified nucleobases, and chemical tools such as fluorinated compounds useful for fluorescent probing and/or chemical labeling/tagging. Doing so may enhance their capability for insertion, expression, and production of functional receptor message for cell member display. Notably, these disclosures may avoid the use of natural forms of genes.

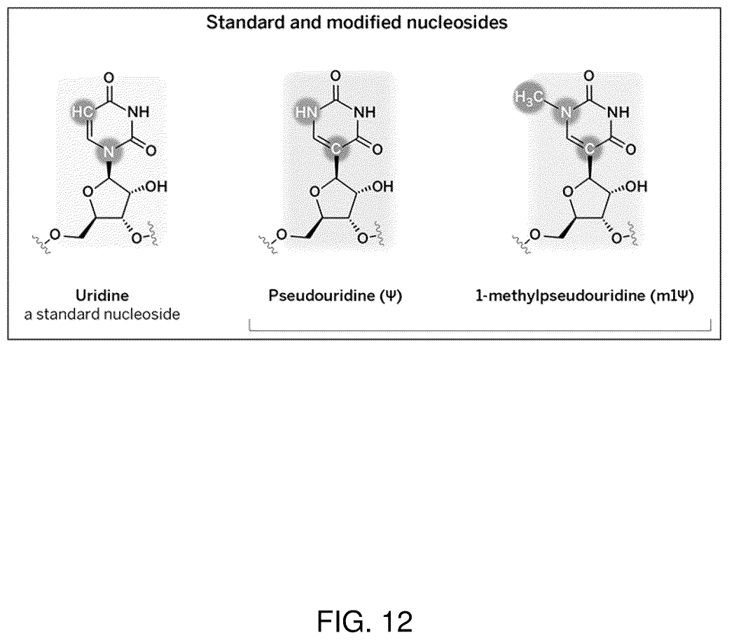

Likewise, regarding the second way of expressing a cell (i.e., delivery and uptake of target cells or tissues that encapsulate mRNA for the receptor), in exemplary embodiments, the selected mRNA is not intended to be native mRNA, but rather nucleoside-modified mRNA (FIG. 12) such that the proficiency of encapsulation, cell uptake, and translation are appropriately encouraged. Specific sequences of these mRNAs may be derived from the coding regions of Homo sapiens genes and sequences, as noted in FIGS. 7-10.

As described herein, the term “delivery method” for the C-type lectin receptor gene or its mRNA may refer to the combinatorial mechanism of:
(a) an infectious non-pathological agent embodying a selected C-type lectin receptor modified gene; or
(b) a target cell-absorbable micellar product (micro- or nanoparticle) encapsulating a functional chemically modified C-type lectin receptor mRNA, wherein the cell mRNA (whether gene expression derived or provided as functional or mRNA) translation produces a functional, cell membrane-insertable functional C-type lectin receptor capable of binding and internalizing an agent (such as TIL-F or FCN).

As described herein, any reference to a “target cell” or “targeted cells”, particularly as it relates to the expression of C-type lectin receptor, is not intended to include germline cells, e.g., sperm or ova.

As described herein, nonpathological modified viral delivery systems may include, but is not limited to, viral vectors such as: retrovirus, adenovirus (types 2 and 5), adeno-associated virus, herpes virus, pox virus, human foamy virus (HFV), lentivirus and poliovirus. All viral vector genomes may be modified by deleting some areas of their genomes so that their replication becomes deranged, thereby rendering them acceptably safe, though adverse effects may be known.

Some viral vectors with specific receptors have been designed with the ability to transfer transgenes to specific cells, which may not include their natural target cells (retargeting). All such systems can provide C-type lectin receptor gene transportation such that the resulting virus-lectin receptor gene may be provided via accepted clinical methodologies, which may include injection (local or systemic), oral delivery, topical delivery, inhalational delivery, suppository delivery, depot delivery, or other acceptable means.

For the purposes of the present invention, mRNA delivery systems may include but are not limited to: (1) monovalent cationic lipids; (2) polyvalent cationic lipids; (3) guanidine-containing lipid formulations; (4) cholesterol derivative compounds; (5) cationic polymers such as poly(ethylenimine) (PEI), poly-l-lysine (PLL), protamine, etc.; and (6) lipid-polymer hybrid mechanisms of gene delivery by cationic particles.

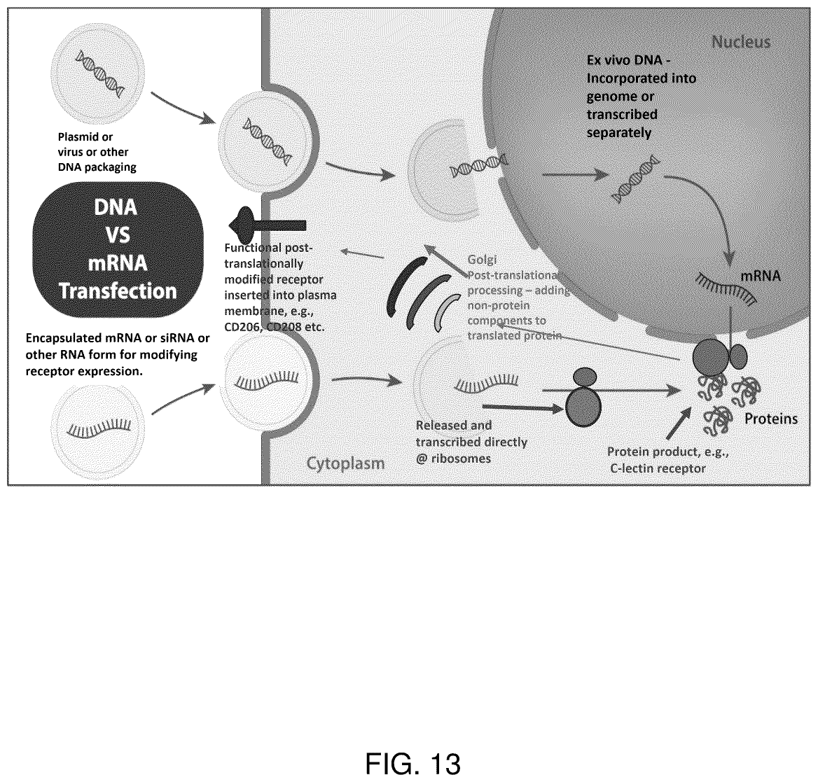

The mechanism of gene delivery by cationic systems includes 4 steps: (1) nonspecific interaction between cationic particles and cell surface; (2) endocytosis into endocytosis vesicles (endosomes); (3) compaction and release of the mRNA particles from endosomes; and (4) transcription of the mRNA particle by ribosomal RNA, glycosylation, packaging, and insertion into the cell membrane. All such systems can provide C-type lectin mRNA transportation such that the resulting virus-lectin receptor mRNA may be provided via accepted clinical methodologies, which may include injection (local or systemic), oral delivery, topical delivery, inhalational delivery, suppository delivery, depot delivery, or other acceptable means. FIG. 13 depicts these expression processes—i.e., the transportation, translocation, and expression of a gene (DNA) or message (mRNA) leading to rough endoplasmic reticulum ribosomal (red arrow) mRNA translation, protein product—golgi-processed (green arrow) with C-type lectin receptor being inserted in the cell membrane (C-lectin receptor).

It is noted that the binding specificity of C-type lectin receptors is based on primary secondary and tertiary carbohydrate structures that may be found on pathogens and/or other antigens. Mannan and mannan derivatives effectively mimic these receptor-perturbing invasive structures.

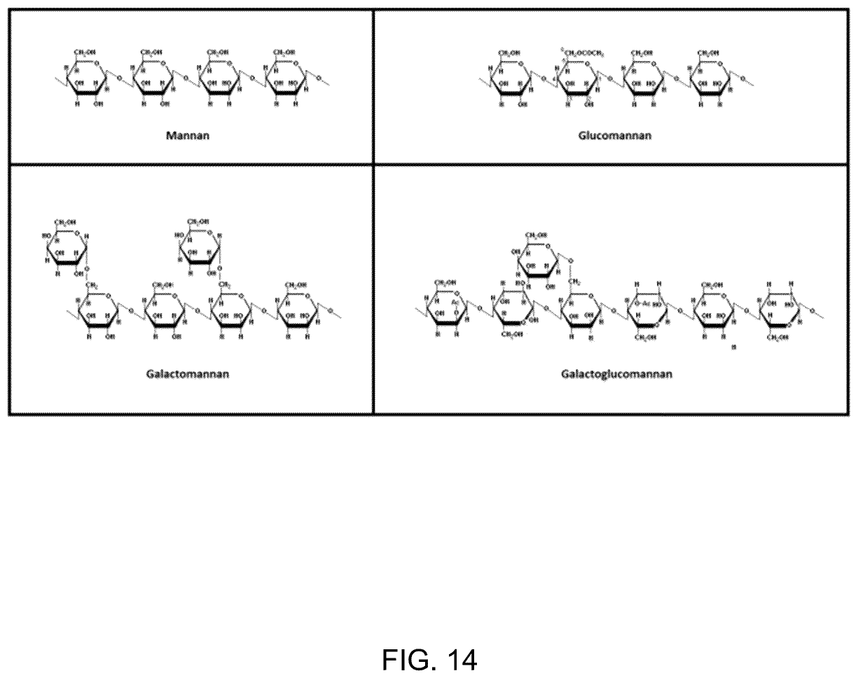

Regarding the induction of the expression of a C-type lectin receptor in target tissue, it is noted that there are cells and tissues that normally express a C-type lectin receptors naturally. Such cells present themselves as possible off target localization of the TIL-F, FCN, or other suitable ligands, resulting in potentially unwanted side effects of treatment. To prevent this, these natural internal sites may be preemptively blocked using a pseudo-ligand such as mannan or one of the various derivatives thereof, such as those shown in FIG. 14. The use of mannan or a derivative thereof as a blocking procedure for localizing therapy to target tissues is an essential element of the present invention.

Another aspect of the present invention relates to compounds that incorporate the cell-suicide inducing agents described above. These compounds may be administered to a subject in need thereof to treat (i.e., cure, ameliorate, stabilize, and/or prevent) a disease or a disorder. This can include, but is not limited to, diseases related to tumor malignancy, dementia, inflammation, arthritis, obesity, leishmaniosis, autoimmune diseases, inflammatory bowel disease (e.g., Crohn's disease), lung disease, viral/bacterial diseases, amyotrophic lateral sclerosis (ALS), multiple sclerosis (MS), and any other disease where CLR-expressing cells are elements of the disease-causing or disease-progressing. This can also include diseases where CLR-expressing cells are elements of the symptomology of a disease, or pathological state, or where the elimination or phenotypic shifting of cells would in some way be advantageous to the host. This can further include any condition where macrophages or other CD206-high expressing cells are involved or recruited, such as when the number of macrophages or other CD206-high expressing cells is increased and/or when these cells are metabolically abnormal.

As it applies to tumors and tumor microenvironments, administering the compound according to the present invention (including all of the variations of the compound described above) to a subject in need thereof may induce cell death in the tumor body or other target cell.

Compounds according to the present invention include TIL-F, FCN, or both, as well as one or more pharmaceutically acceptable carriers. As used herein, the term “pharmaceutically acceptable carrier” may refer to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity may be maintained, for example, by the use of coating materials such as lecithin, and by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol, sorbic acid and the like. It may additionally or alternatively be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release may be controlled. Depot injectable formulations are also prepared by entrapping the drug in liposomes or microemulsions which are compatible with body tissues. The injectable formulations may be sterilized, for example, by filtration through a bacterial-retaining filter or by incorporating sterilizing agents in the form of sterile solid compositions which may be dissolved or dispersed in sterile water or other sterile injectable media just prior to use. Suitable inert carriers may include sugars such as lactose. Desirably, at least 95% by weight of the particles of the active ingredient have an effective particle size in the range of 0.01 to 10 micrometers.

Other additives which may be included in compounds according to the present invention include, but are not limited to: anions such as bromide (Br-1), nitrate (NO-3), and thiocyanate (SCN-1); isoflavones such as rotenone; dinitrophenols such as 2,4-dinitrophenol (DNP); free fatty acids such as decanoate and octanoate; mitochondria-specific fluorescent probes such as MitoFluo; and mitochondria-targeted protonophores such as Mito-Photo-DNP.

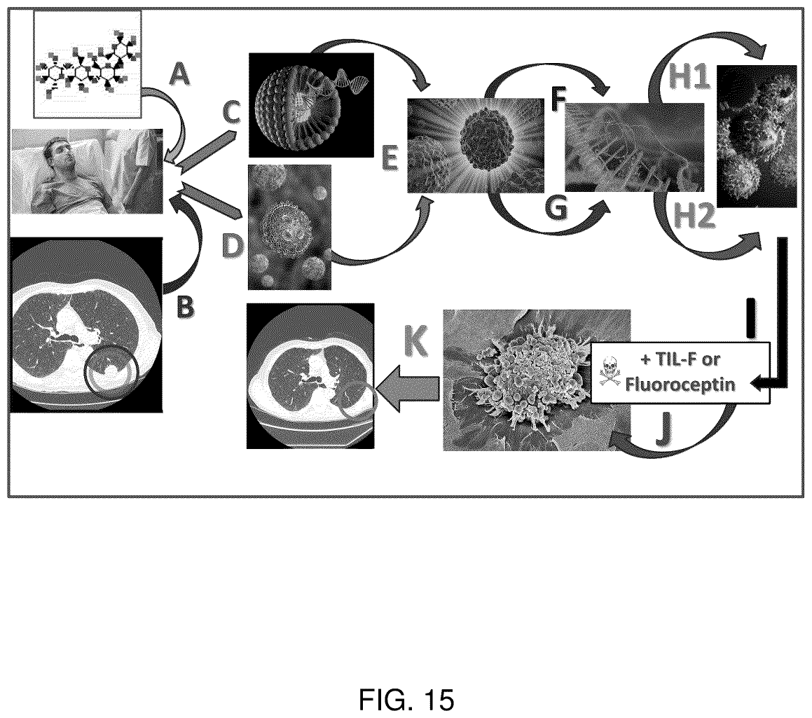

In yet another aspect of the present invention, provided are methods for the treatment of diseases and/or disorders using compounds according to the present invention. Depicted in FIG. 15 is the overall schema of such methods, which may include any one or more of: A] Predosing with mannan or suppressing off target binding with siRNA; B] establishing the target, in this case a tumor (circled); C Delivering the up-regulation of the receptor by transcriptable DNA fragment via virus or encapsulation; D] delivering a functional RNA, e.g. mRNA via encapsulation (resulting in membrane fusion and release of cytoplasm in cell of receptor RNA; E] Functional mRNA (either via DNA transcription or provided from ex vivo; F] Via DNA; G] via provided mRNA; H1/H2] Translation: post-translationmodification: insertion; I] suicide TIL-F provided, binding to C-lectin receptor; J] Internalized and induction of suicide (apoptosis); K] Tumor elimination with little of no clinically significant off target effect.

FIG. 15 is best read from left to right and can be generally described as follows:
1. A patient expressing a particular tumor (B) (depicted as a lung tumor) is preemptively dosed with mannan (A).
2. The patient is then dosed with either the receptor gene (C) (carried via a viral vector, for example) or is dosed with encapsulated mRNA (D) by a selective exposure route (e.g., inhalation, systemic injection, local/direct injection, or other clinically means adapted to the anatomical location).
3. The tumor (E) takes up the genetic material via viral infection or incorporates the encapsulated mRNA within the tumor cells to express the C-type lectin receptor. In the case of gene transfection, this process involves transcription/translation while in the case of the mRNA only translation is necessary (F and G, respectively).
4. Regardless of the route of receptor production (H1 or H2) the C-type lectin receptor is now expressed on the surface of the tumor cells (by the message which is DNA derived or provided-mRNA and processed via translation and post-translational modification prior to cell membrane insertion), making the tumor vulnerable to attack by either TIL-F or FCN (I).
5. Exposure to either TIL-F or FCN results in the induction of apoptosis and tumor cell death (J).
6. After treatment, evaluation of the tumor site may yield the absence of tumor as (K) (depicted by the CT scan).

The present invention significantly improves therapeutic approaches to multiple diseases by employing at least one of TIL-F and FCN, both of which are targeted primarily to CD206. It is noted that CD205, CD208, and/or other C-type lectin receptors may be secondarily targeted as well.

In some embodiments, TIL-F and/or FCN may have modified terminal carbohydrate moieties that accommodate different C-type lectin receptors (other than CD206). As previously stated, C-type lectin receptors' recognition affinities are dependent upon structural carbohydrate moieties of the ligand. Some other carbohydrate moieties that may be suitable for this role can include, but are not limited to, galactose, fucose, n-acetyl glucosamine, as well as other pentose and hexose moieties. The C-type lectin receptor genes and messenger RNA noted in Table 1 and FIG. 6 may be utilized as substitutes for the CD206 gene and/or its messenger RNA as the functional therapeutic material outlined in the method of FIG. 15.

Appropriate dosage amounts for the compounds according to the present invention (including all variations of the compound described above) may vary. Appropriate dosage amounts are likely to be determined based on factors such as BMI, disease, administration selection, anatomic location, and pharmacokinetics and pharmacodynamics of the drug. In exemplary embodiments, the dosage amount may fall within a range from about 0.001 milligrams to about 50 grams over a single 24-hour period, or more preferably from about 0.001 milligrams to about 10 grams over a single 24-hour period, or even more preferably from about 0.001 milligrams to about 2 grams over a single 24-hour period. Other ranges and/or amounts may be utilized.

Although the foregoing description is directed to the preferred embodiments of the invention, it is noted that other variations and modifications will be apparent to those skilled in the art and may be made without departing from the spirit or scope of the invention. Moreover, features described in connection with one embodiment of the invention may be used in conjunction with other embodiments, even if not explicitly stated above.

The invention is not limited in its application to the details of construction and the arrangement of components set forth in the following description or illustrated in the following drawings. The invention is capable of other embodiments and of being practiced or of being carried out in various ways.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. A cell suicide-inducing agent comprising:
a dextran backbone;
at least one linker attached to the dextran backbone;
a C-type lectin targeting moiety conjugated to a linker; and
at least one of:
at least one fluoride ion covalently bonded to the dextran backbone or a linker; and
an anion chelating agent conjugated to a linker and an anion bonded to the anion chelating agent.

2. The agent of claim 1, wherein:
the C-type lectin targeting moiety comprises at least one of mannose, galactose, fucose, n-acetyl glucosamine, pentose, and hexose.

3. The agent of claim 1, wherein:
the agent comprises between 1 and 250 fluoride ions which are, on an individual basis, covalently bonded to the dextran backbone or a linker.

4. The agent of claim 1, wherein:
the at least one linker comprises a bifunctional amino-terminated sulfide linker.

5. The agent of claim 4, wherein:
the distal end of the bifunctional linker comprises a primary carbon that is fluorinated with 1 or 2 fluoride ions.

6. The agent of claim 1, wherein the agent comprises:
at least one fluoride ion covalently bonded to the dextran backbone or a linker; and
at least one chelated cation.

7. The agent of claim 6, wherein:
the agent comprises between 1 and 250 chelated cations.

8. The agent of claim 6, wherein:
at least one of the cations is chelated by diethylenetriamine pentaacetic acid (DTPA).

9. The agent of claim 1, wherein:
an anion chelating agent conjugated to a linker and an anion bonded to the anion chelating agent; and
the anion is a monovalent anion such as fluorine.

10. The agent of claim 9, wherein:
the anion chelating agent comprises at least one of:
a macrocycle, preferably 1,2,3-triazole-containing calixarenes;
a distiborane, preferably distiboranes with the general formula o-C₆F₄(SbPh₂(diolate))₂, with the diolate being tetrachlorocatecholate or octafluorophenanthrene-9,10-diolate; and
salicylidene-tris(aminomethyl)ethane (SALTAME).

11. The agent of claim 1, further comprising:
a stabilizing group attached to a linker, the stabilizing group comprising at least one of methyl acrylate and N-ethylmaleimide.

12. A compound for the elimination of living vertebrate tissue comprising:
the cell suicide-inducing agent of claim 1; and
a pharmaceutically acceptable carrier.

13. A method for the treatment of diseases or disorders, the method comprising:
administering, to a subject, a dose of mannan or a derivative thereof;
administering, to the subject, at least one of:
a dose of a substance containing a receptor gene; and
a dose of a substance containing encapsulated mRNA for a designated receptor; and
administering, to the subject, a dose of the compound of claim 12.

14. The method of claim 13, wherein:
the dose of mannan or a derivative thereof comprises at least one of: mannan, glucomannan, galactomannan, and galactoglucomannan.

15. The method of claim 13, wherein:
the method comprises administering, to the subject, a dose of a substance containing a receptor gene for a C-Type lectin receptor, preferably at least one of: CD205, CD206, MRC1, LAMP3, CD209, and MRC2.

16. The method of claim 13, wherein:
the method comprises administering, to the subject, a dose of a substance containing a receptor gene; and
the receptor gene has been modified with at least one of the following: iso-G, iso-C, 5SICS, MMO2, Ds, Pa, ^{F}I, and ^{F}B.

17. The method of claim 13, wherein:
a nonpathological modified viral delivery system is used to deliver a dose of a substance containing a receptor gene, the viral delivery system comprising at least one of: retrovirus, adenovirus (type 2), adenovirus (type 5), adeno-associated virus, herpes virus, pox virus, human foamy virus (HFV), lentivirus and poliovirus; and
one or more areas of the genome of the viral delivery system has been deleted such that their replication becomes deranged.

18. The method of claim 13, wherein:
the method comprises administering, to the subject, a dose of a substance containing encapsulated mRNA for a designated receptor; and
the mRNA comprises at least one of the following modified nucleosides: pseudouridine (ψ) and 1-methylpseudouridine (m1ψ).

19. The method of claim 13, wherein:
a mRNA delivery system is used to administer a dose of a substance containing encapsulated mRNA for a designated receptor, the mRNA delivery system comprising at least one of:
monovalent cationic lipids;
polyvalent cationic lipids;
guanidine-containing lipid formulations;
cholesterol derivative compounds;
cationic polymers, preferably poly(ethylenimine) (PEI), poly-l-lysine (PLL), and protamine; and
lipid-polymer hybrid mechanisms of gene delivery by cationic particles.

20. The method of claim 13, wherein:
the dose of the compound of claim 12 is in an amount ranging from about 0.001 milligrams to about 50 grams over a single 24-hour period, or more preferably from about 0.001 milligrams to about 10 grams over a single 24-hour period, or even more preferably from about 0.001 milligrams to about 2 grams over a single 24-hour period.

21. The method of claim 13, wherein:
the step of administering, to the subject, at least one of a dose of a substance containing a receptor gene and a dose of a substance containing encapsulated mRNA for a designated receptor is performed by at least one of local injection, systemic injection, oral delivery, topical delivery, inhalational delivery, suppository delivery, and depot delivery.
